(19) United States
(12) Patent Application Publication
(10) Pub. No.: US 2015/0313464 A1
(43) Pub. Date: Nov. 5, 2015

(54) LENS HANDLE WITH ROTATIONAL CONTROL

(71) Applicant: Ocular Instruments, Inc., Bellevue, WA (US)

(72) Inventor: Raymond D. Graham, Renton, WA (US)

(73) Assignee: OCULAR INSTRUMENTS, INC., Bellevue, WA (US)

(21) Appl. No.: 14/703,445

(22) Filed: May 4, 2015

Related U.S. Application Data
(60) Provisional application No. 61/987,678, filed on May 2, 2014.

(51) Int. Cl. A61B 3/117; G02B 7/02; A61B 3/00

(52) U.S. Cl. CPC A61B 3/117; A61B 3/0075; G02B 7/026; G02B 7/023

(57) ABSTRACT

A lens handle assembly of one embodiment of the present disclosure includes a handle; a lens carried by the handle in a manner that provides rotation thereof; and an actuator carried by the handle, wherein the actuator is configured to affect rotation of the lens.

### CROSS-REFERENCE TO RELATED APPLICATION

This application claims the benefit of U.S. Provisional Application No. 61/987,678, filed May 2, 2014, the disclosure of which is hereby expressly incorporated by reference in its entirety.

### SUMMARY

This summary is provided to introduce a selection of concepts in a simplified form that are further described below in the Detailed Description. This summary is not intended to identify key features of the claimed subject matter, nor is this summary intended to be used as an aid in determining the scope of the claimed subject matter.

The present disclosure relates generally to instruments of the type broadly applicable to numerous medical procedures of the eye. As will be described in more detail below, one or more examples of a medical instrument in accordance with aspects of the present disclosure include a lens handle that provides the surgeon with the capability of viewing the entire 360 degree of the anterior chamber angle (ACA) while employing an “on-axis” style viewing lens (e.g., a direct viewing lens, a mirrored lens, etc.) In various embodiments shown and described herein, the lens handle allows the surgeon to both hold the lens in an “on axis” orientation and to manipulate (e.g., rotate) the lens about the optical axis of the patient's eye so as to see the entire 360 degree of the ACA with the same hand (i.e., a “one-handed operation”).

In accordance with one embodiment of the present disclosure, a lens handle assembly is provided. The assembly includes a handle; a lens carried by the handle in a manner that provides rotation thereof; and an actuator carried by the handle, wherein the actuator is configured to affect rotation of the lens.

In accordance with another embodiment of the present disclosure, a lens handle assembly is provided. The assembly includes a handle; a lens carried by the handle in a manner that provides rotation thereof; and means for affecting rotation of the lens.

In accordance with one embodiment of the present disclosure, a method of viewing the ACA of a patient is provided. The method includes providing a lens handle assembly including a handle, a lens carried by the handle in a manner that provides rotation thereof, and an actuator carried by the handle, wherein the actuator is configured to affect rotation of the lens; holding the handle of the lens handle with one hand; and rotating the lens assembly with the one hand via the actuator.

In any of the embodiments described herein, the lens may be surrounded by a collar, the collar defining a ring gear.

In any of the embodiments described herein, the lens handle assembly of claim 1, wherein the actuator includes a drive shaft having a drive gear disposed on the distal end thereof, the drive gear configured and arranged to mesh with the ring gear.

In any of the embodiments described herein, the actuator may be manually actuated.

In any of the embodiments described herein, the actuator may be actuated via a drive motor.

In any of the embodiments described herein, the drive motor may be mounted to the handle and interfaces with the drive shaft.

In any of the embodiments described herein, the lens handle may include a handle portion and a lens retainer portion.

### DESCRIPTION OF THE DRAWINGS

The foregoing aspects and many of the attendant advantages of the disclosed subject matter will become more readily appreciated as the same become better understood by reference to the following detailed description, when taken in conjunction with the accompanying drawings, wherein:

FIG. 1A is a top perspective view of one example of a lens handle in accordance with aspects of the present disclosure;

FIG. 1B is a bottom perspective view of the lens handle of FIG. 1A;

FIG. 2 is an exploded side view of the lens handle of FIG. 1A, depicted one example of a handle, and a lens assembly comprises a lens housing and a lens;

FIG. 3 is a top perspective view of one example of a lens housing formed in accordance with aspects of the present disclosure;

FIG. 4 is a bottom perspective view of the lens housing of FIG. 3;

FIG. 5 is a top plan view of the lens housing of FIG. 3;

FIG. 6 is a cross-sectional view of the lens housing taken along lines 6-6 in FIG. 5;

FIG. 7 is a side cross-sectional view of one example of the handle shown in FIG. 2;

FIG. 8A is a top perspective view of another example of a lens handle in accordance with aspects of the present disclosure;

FIG. 8B is a bottom perspective view of the lens handle of FIG. 8A;

FIG. 9 is an exploded view of the lens handle of FIG. 8A;

FIG. 10 is a top plan view of the lens housing of FIG. 9;

FIG. 11 is a cross-sectional view of the lens housing taken along lines 11-11 in FIG. 10;

FIG. 12 is the cross-sectional view of the lens housing of FIG. 11 with a lens retained therein; and

FIG. 13 is a block diagram depicting one example of a control system for a drive motor in accordance with aspects of the present disclosure.

### DETAILED DESCRIPTION

The detailed description set forth below in connection with the appended drawings where like numerals reference like elements is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the claimed subject matter to the precise forms disclosed.

The following discussion relates generally to instruments suitable for use in various medical procedures of the eye. In particular, the following discussion provides examples of a lens handle that can be used during treatment of, for example, glaucoma. Examples of the lens handle include a lens assembly carried by a handle, and a means by which manipulation of the lens assembly with respect to the handle can be realized. As will be described in more detail below, some examples of the lens assembly can be manipulated about the optical axis of the patient's eye so as to see the entire 360 degree of the anterior chamber angle (ACA). The examples described herein provide a medical instrument with various benefits over the prior art, including but not limited to improved field of vision, increased field of vision by rotation (not limited to the static field), improved ergonomic control of the lens by the doctor, and user control of view location, dynamically, thereby eliminating the need to “look away” from the microscope to rotate the lens.

In the following description, numerous specific details are set forth in order to provide a thorough understanding of one or more embodiments of the present disclosure. It will be apparent to one skilled in the art, however, that many embodiments of the present disclosure may be practiced without some or all of the specific details. In some instances, well-known process steps have not been described in detail in order not to unnecessarily obscure various aspects of the present disclosure. Further, it will be appreciated that embodiments of the present disclosure may employ any combination of features described herein.

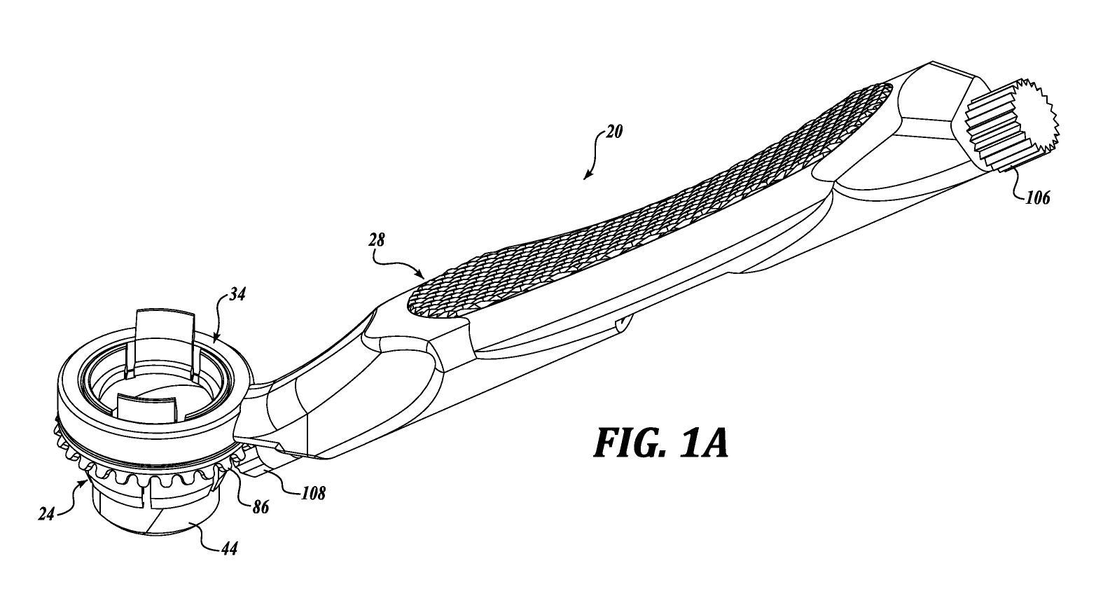

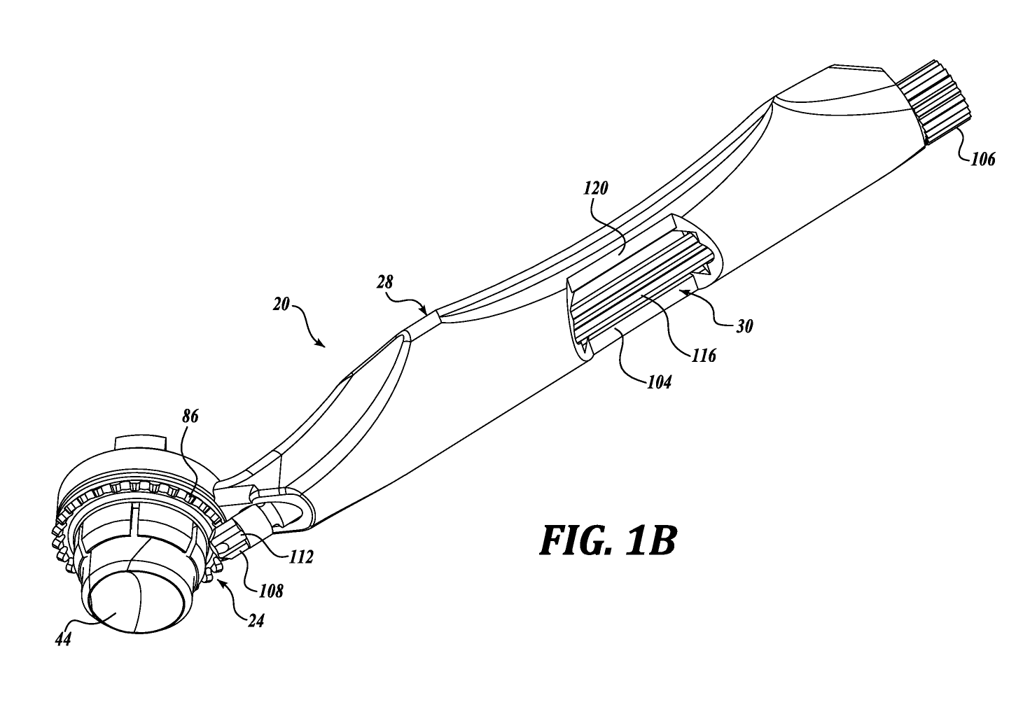

Turning now to FIGS. 1A-1B, there is shown one example of a lens handle, generally designated 20, formed in accordance with aspects of the present disclosure. The lens handle 20 is suitable for use during medical procedures of the eye, such as for example, the treatment of glaucoma or the like. Generally described, the lens handle 20 includes a lens assembly 24 carried by or otherwise associated with a handle 28. As will be described in more detail below, the lens handle 20 is configured for one-handed operation, including a user manipulatable actuator 30 (see FIG. 1B) that affects movement of the lens assembly 24 with respect to the handle 28. In use, the lens handle 20 can be grasped with one hand of the user while the other hand of the user is free to hold another instrument associated with the particular medical procedure. While the lens handle 20 is in the hand of the user, the lens assembly 24 can be manipulated firstly by movement of the handle 28 via the user's wrist or arm, and secondly, by actuation of the actuator 30 with the user's finger or fingers of the hand grasping the handle 28.

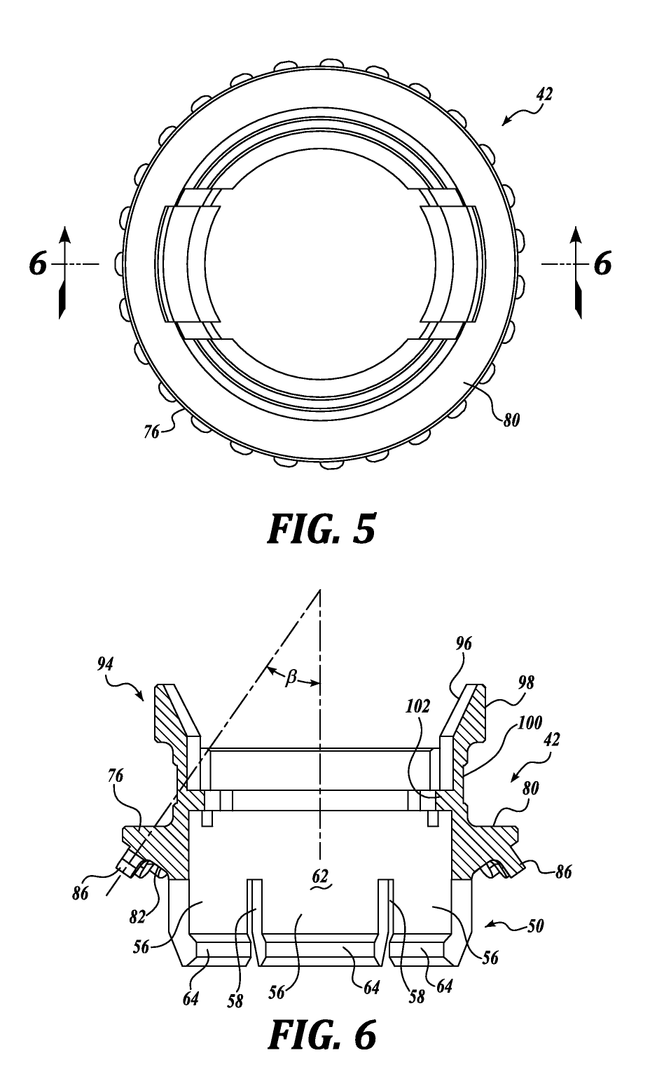

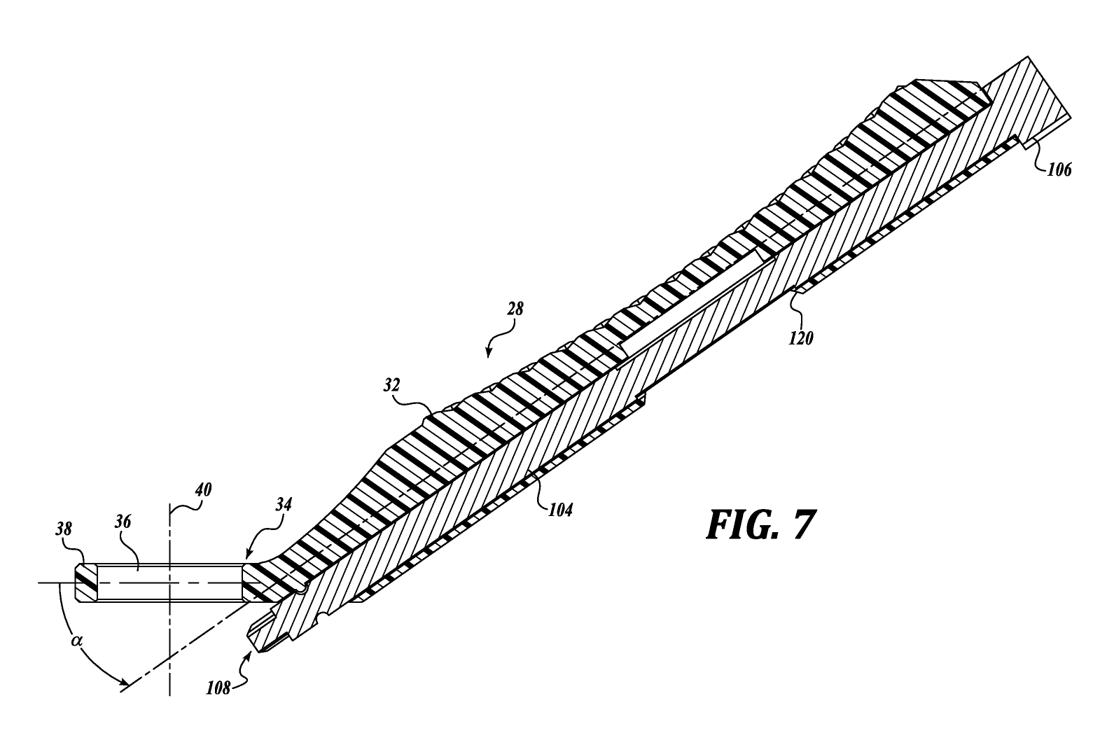

Referring to FIGS. 1-7, the components of the lens handle 20 will be described in more detail. As shown in FIGS. 1A-1B, 2, and 7, the lens assembly 24 is carried at the end of the handle 28. In that regard, the handle 28 includes an elongate body 32 to which a lens assembly retainer 34 is formed, attached, or otherwise provided at the distal end thereof. In the embodiment shown in FIGS. 1A and 7, the lens assembly retainer 34 is in the form of a ring defining a cylindrical bore 36 (FIG. 7), and having walls 38 with a generally rectangular cross section and a top chamfered edge. The lens assembly retainer 34 is disposed at an angle α with respect to the longitudinal axis of the handle 28, as shown in FIG. 7. In some embodiments, the angle α is approximately between 30 and 40 degrees or greater, and in one embodiment, is approximately 35 degrees. In other embodiments, the angle α is approximately between 0-15 degrees or greater for use with, for example, slitlamp lenses. As such, one or more embodiments may employ an angle α approximately between 0-50 degrees. As will be described in more detail below, the lens assembly retainer 34 is sized and configured to interface with the lens assembly 24 for releasable securement therewith. Once coupled, the lens assembly 24 is allowed to rotate about the axis 40 of the bore 36.

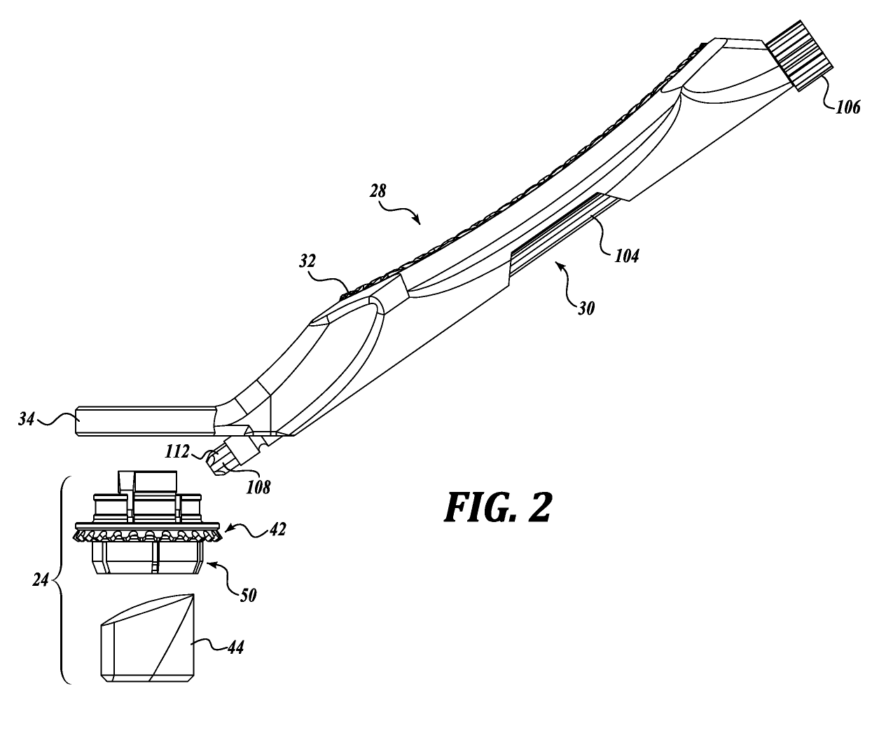

Referring now to FIG. 2, the lens assembly 24 in some embodiments includes a collar-like lens housing 42 (“lens housing 42”) that surrounds a lens 44. The lens 44 can be any suitable “on-axis” style viewing lens (e.g., a direct viewing lens, a mirrored lens, etc.). In one embodiment, the lens can be a 4-mirrored lens. In another embodiment, the lens can be either a three or four mirrored lens of universal design for viewing the ACA and the posterior fundus and retina. The lens in other embodiments may be of the prism type.

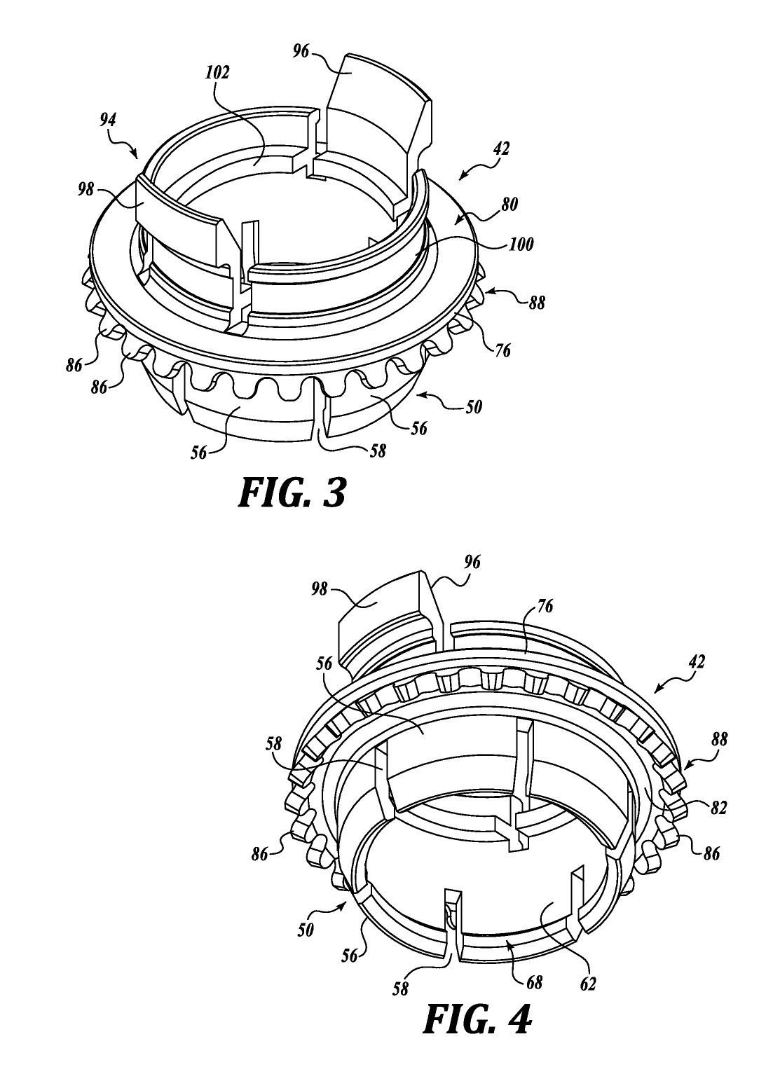

At its distal end, the lens housing 42 includes a lens retaining interface 50 configured to retain or hold the lens 44 in position during use. In some embodiments, the lens retaining interface 50 is in the form of a collet having a plurality of annularly disposed legs 56 separated by kerfs or slots 58, as shown in in FIGS. 3, 4, and 6. The collet defines a generally cylindrical, inner cavity 62 for receiving at least a portion of the lens 44 therein. As shown in FIG. 6, the outer, free ends of the legs 56 can be slanted generally inwardly in some embodiments, each forming an engagement flange segment 64. Together, the engagement flange segments 64 define the distal opening 68 (see FIG. 4) of the lens housing 42, which communicates with the inner cavity 62.

In some embodiments, the legs 56 are configured and arranged to slightly flex outwardly during installation of the lens 44. As a result, the engagement flange segments 64 of the slightly flexed legs 56 apply pressure to the outer surface of the lens 44. This pressure, along with frictional forces between the lens 44 and the inner walls of the housing 42, releasably retain or hold the lens 44. In the embodiment shown in FIGS. 1A-1B, a portion of the lens 44 extends outwardly of the distal end of the lens housing 42 once retained by the collet of the lens housing 42. It will be appreciated that the lens 44 can be any type of lens useful in one or more surgical procedures, including but not limited to a direct viewing lens, a mirrored lens, etc.

Returning to FIGS. 3, 4, and 6, the lens housing 42 also includes an annular flange 76 spaced proximally of the lens retaining interface 50 (e.g., collet, etc.). The annular flange 76 extends radially outwardly of the housing 42, and in some embodiments, has a somewhat truncated, right triangular-like cross section (FIG. 6). In that regard, the flange 76 defines a proximal facing surface 80 positioned orthogonal to the longitudinal axis of the housing 42 and a slanted surface 82. Extending from the slanted surface 82 of the flange 76 are a plural of gear teeth 86, thereby forming a ring gear 88 (see FIGS. 3 and 4). In the embodiment shown, the ends of the teeth 86 of the ring gear 88 are generally rounded and extend at an angle β with respect to the longitudinal axis of the housing 42 (see FIG. 6). In some embodiments, the angle β is approximately between 30-40 degrees, and in one embodiment, is approximately 35 degrees. In these and other embodiments, the angle β is approximately equal to the angle α.

The lens housing 42 further includes a handle coupling interface 94 disposed at its proximal end, opposite the lens retaining interface 50. The handle coupling interface 94 is configured to couple the lens housing 42 with the lens assembly retainer 34 of the handle 28. In some embodiments, the handle coupling interface 94 is configured to releasably couple the lens housing 42 to the handle 28.

In the embodiment shown in FIGS. 3, 4, and 6, the handle coupling interface 94 includes a pair of opposing snap retainers 96 extending in the proximal direction from the outer annular walls of a proximal section of the lens housing 42. The snap retainers 96 include radially outwardly extending flange sections 98, the bottoms of which form an annular channel 100 with the proximal facing surface 80 of the annular ring 76. The snap extensions 96 are configured and arranged to slightly flex inwardly during coupling of the lens assembly 24 to the handle 28. In that regard, the flange segments 98 snap back (with the snap extensions) after they pass through the bore 36 of the lens assembly retainer 34, causing the lens assembly retainer 34 to be disposed in the channel 100 and surrounding the lens housing 42, and thus, coupling the lens assembly 24 to the handle 28. Once coupled, the lens assembly 24 is allowed to rotate with respect to the handle 28 about the longitudinal axis 40 of the bore 36. In use, the longitudinal axis 40 is generally aligned with the optical axis of the patient's eye.

In some embodiments, the lens housing 42 includes an optional, inner annular flange 102 positioned somewhat in the proximal cavity. The inner annular flange 102 in some embodiments may be used as an end stop for insertion of the lens 44.

Returning now to FIGS. 1A-1B and 7, the actuator 30 is carried by the body 32, and is configured and arranged to interface with the lens assembly 24 in order to manipulate the lens assembly 24. In the embodiment shown in FIG. 7, the actuator 30 includes a drive shaft 104 journaled for rotation about an axis parallel with the longitudinal axis of the handle 28. At the distal end of the drive shaft 104 there is formed, attached or otherwise provided a drive gear 108. The drive gear 108 includes a plurality of teeth 112 configured and arranged to cooperate with the teeth 86 of the ring gear 88 such that rotation of the drive shaft 104 results in rotation of the lens assembly 24. Along the length of the body, the drive shaft 104 can include a lever in the form of a knob or can be formed with a splined or knurled section to interface with a finger or fingers of the user. In that regard, the handle body 32 may include a recess 120 or the like to provide access to the drive shaft 104 in order for the user's finger to contact and rotate the drive shaft 104. Access to the drive shaft 104 is positioned in an ergonomic location such that the user (e.g., surgeon) can hold the handle 28 and rotate the drive shaft in a one handed operation. The handle body 32 in some embodiments may be ergonomically configured for comfort when gripped by the doctor and can include one or more knurled surface sections.

Additionally or alternatively, the drive shaft 104 may include an enlarged knob 106 formed, affixed, mounted, or otherwise disposed at the proximal end thereof. In several embodiments of the present disclosure, the knob 106 provides an alternative or additional lever suitable for use by the doctor in order to rotate the drive shaft 104.

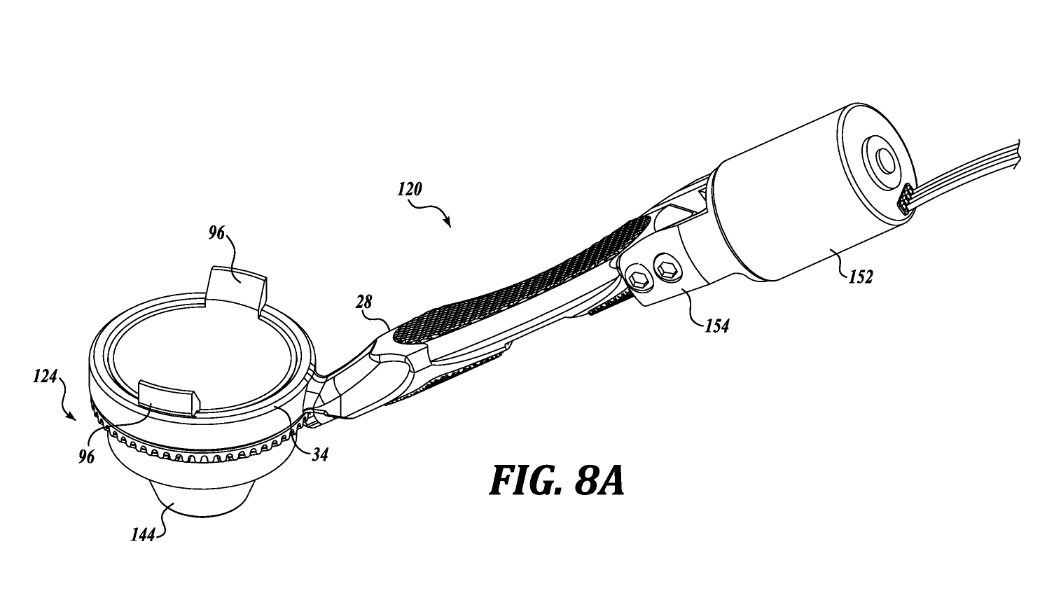

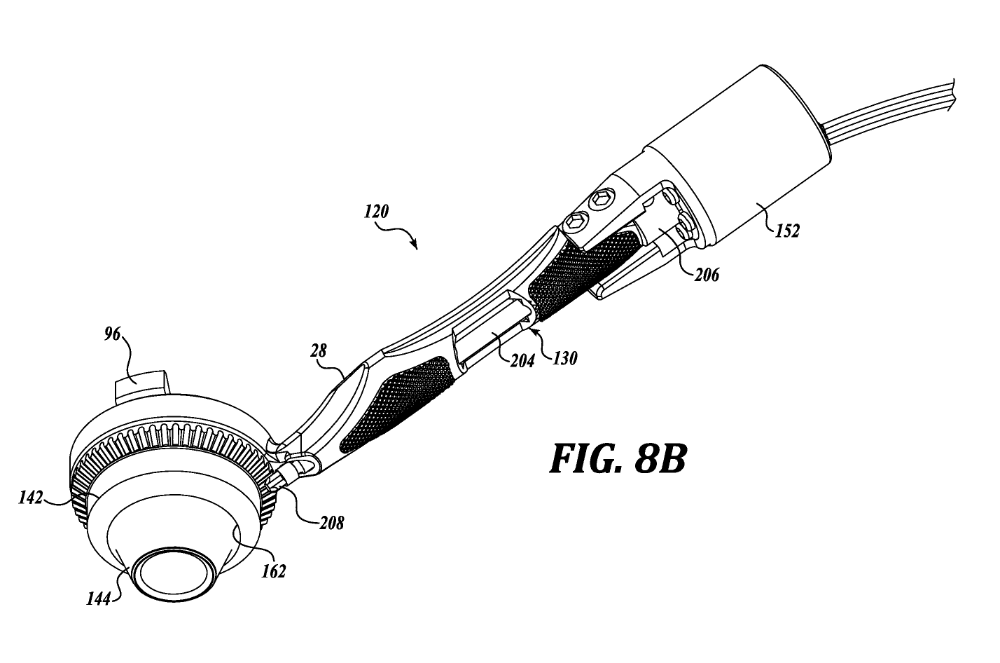

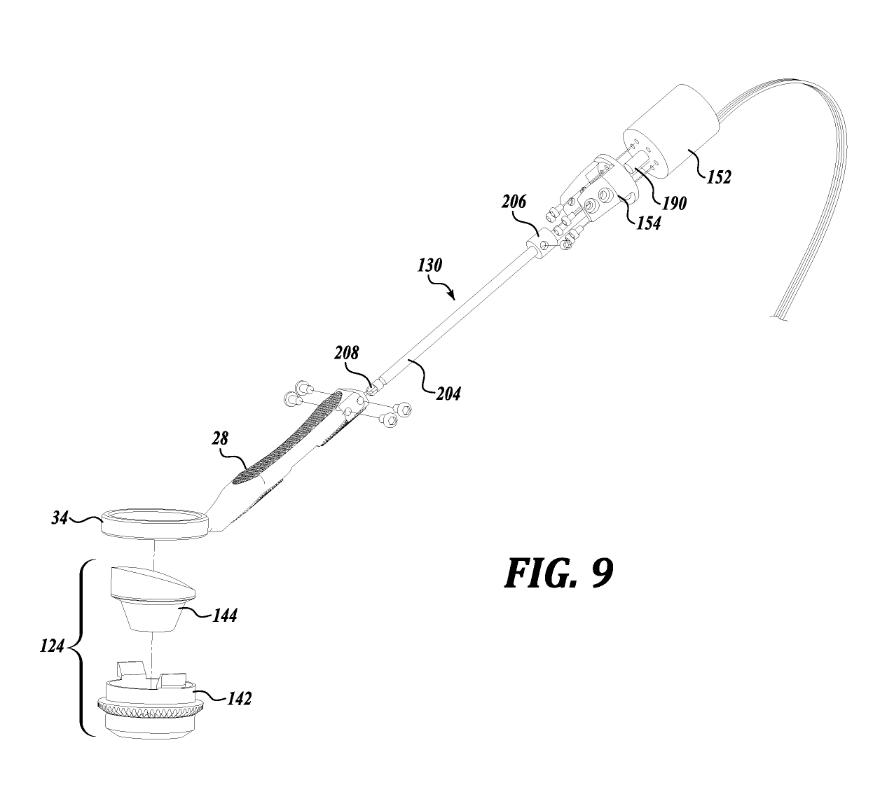

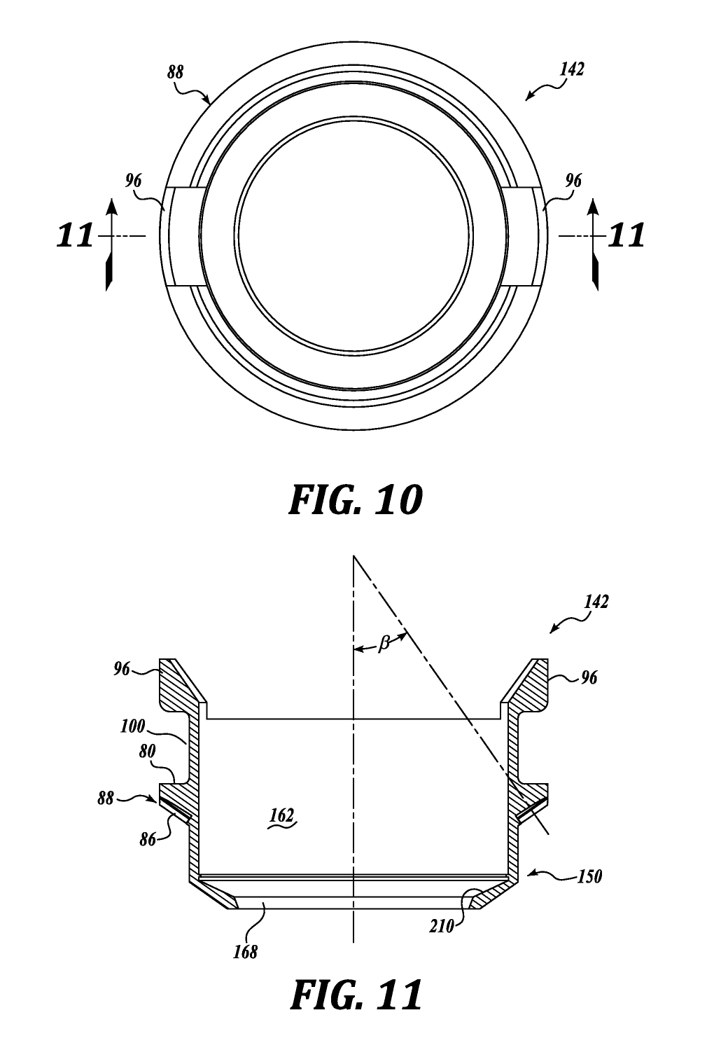

FIGS. 8A-8B illustrate another embodiment of a lens handle 120 in accordance with aspects of the present disclosure. The lens handle 120 is substantially identical to lens handle 20 described above with reference to FIGS. 1A-7 except for the differences that will now be described. In that regard, attention is directed to FIGS. 8A-13, which illustrates one example of a lens handle 120 in which the actuator 130 is driven by a drive motor 152. As best shown in FIGS. 8B and 9, the drive motor 152 is mounted to the proximal end of the handle body 32 via mounting bracket 154 or other suitable structure. The drive motor 152 includes an output shaft 190 that is configured to interface (e.g., keyed, splined, pinned, etc.) with the knob 206 of the drive shaft 204 for effecting co-rotation therebetween. While the output shaft 190 is oriented coaxially with the drive shaft 204, other configurations are possible. For example, in some embodiments, the output shaft 190 can be offset with the drive shaft 204 or can be disposed orthogonal thereto, etc.

Drive signals for operating the drive motor 152 with either continuous or incremental rotation can be supplied via activation of a switch 194. The switch 194 can be mounted on the handle 28 or remote therefrom, such as a foot switch, table mounted switch, etc. As such, activation of the switch 194, such as by movement, delivers device specific control signals to be carried out by the drive motor 152. In some embodiments, the drive motor 152 can include but is not limited to AC or DC electric motor, a stepper motor, a servo motor, etc.

In one embodiment, the drive motor 152 includes a stepper motor that receives signal pulses from a controller 196, such as a microcontroller, via operation of the switch 194. The stepper motor can be servo-controlled, depending on its intended application. In response to the signal pulses, the stepper motor rotates the output shaft 190 clockwise/counterclockwise, in increments or “steps” of full shaft rotation. In turn, the output shaft 190 drives the drive shaft 204 in order to rotate the lens assembly 124 from 0-90 degrees in some embodiments (e.g., using a 4-mirrored lens, etc.), and between 0-360 degrees in other embodiments.

The lens handle 120 also employs another example of a lens housing, generally designated 142. The lens housing 142 can also be employed with the handle 28 described above. In that regard, various configurations of the lens housing may be employed with the lens handles 20, 120 depending on its intended application (e.g., which lens 44 is preferred by the doctor for a given ophthalmological procedure). In that regard, any lens housing that either permanently or selectively retains a lens 44 while also providing a suitable interface with the actuator may be practiced with embodiments of the present disclosure.

As shown in FIGS. 9-11, the lens housing 142 is configured for use with, for example, a prism lens 144 (See FIG. 9). In that regard, the lens housing 142 is generally collar shaped for retaining the lens 144. The lens housing 142 includes a handle coupling interface 194 configured to couple the lens housing 142 to the lens assembly retainer 34 of the handle 28. In some embodiments, the handle coupling interface 194 is configured to releasably couple the lens housing 142 to the handle 28.

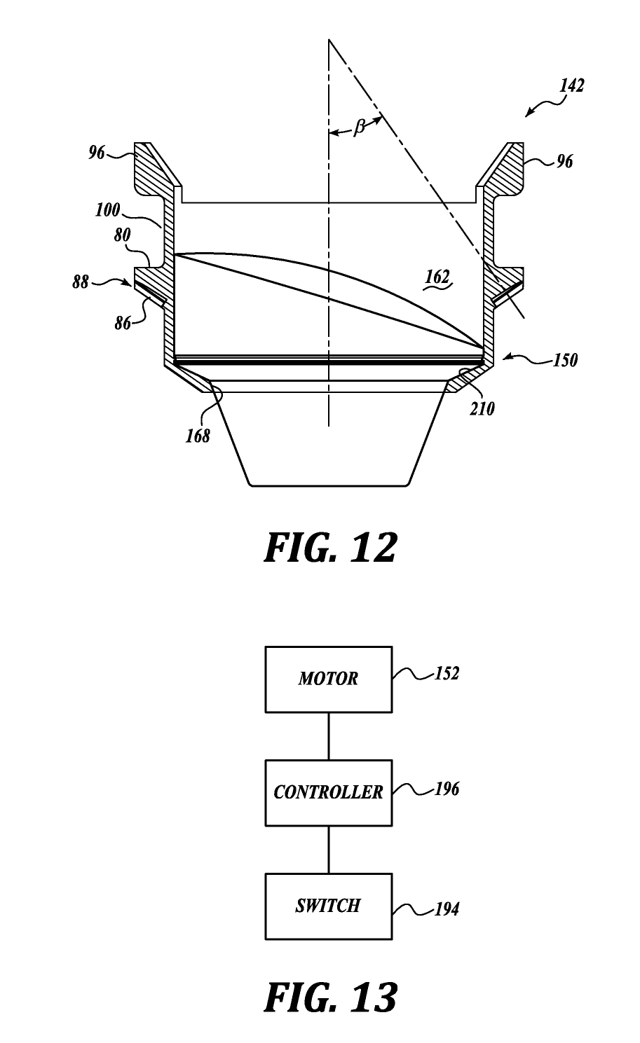

At its distal end, the lens housing 142 includes a lens retaining interface 150 configured to retain or hold the lens 144 in position during use. In some embodiments, the lens retaining interface 150 forms of an internal shoulder 210 formed by a distal opening 168 of smaller cross section than the interior cavity 162 of the main body of the lens housing 142. The shoulder 210 and opening 168 cooperatively receive the lens 144 when assembled, as shown in FIG. 12.

While one example of a gear arrangement has be illustrated and described, it will be appreciated that other rotary to rotary mechanisms may be employed in embodiments of the lens handle 20, 120. Additionally, other actuators that provide rotation to the lens assembly may be practiced with embodiments of the present disclosure. For example, actuators employing reciprocating to rotary mechanisms, etc., to rotate the lens assembly 24, 124 may be used.

It should be noted that for purposes of this disclosure, terminology such as “upper,” “lower,” “vertical,” “horizontal,” “fore,” “aft,” “inner,” “outer,” “inwardly,” “outwardly,” “proximal”, “distal,” “front,” “rear,” etc., should be construed as descriptive and not limiting the scope of the claimed subject matter. Further, the use of “including,” “comprising,” or “having” and variations thereof herein is meant to encompass the items listed thereafter and equivalents thereof as well as additional items. Unless limited otherwise, the terms “connected,” “coupled,” and “mounted” and variations thereof herein are used broadly and encompass direct and indirect connections, couplings, and mountings.

The principles, representative embodiments, and modes of operation of the present disclosure have been described in the foregoing description. However, aspects of the present disclosure which are intended to be protected are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

The embodiments of the disclosure in which an exclusive property or privilege is claimed are defined as follows:

1. A lens handle assembly, comprising:
a handle;
a lens carried by the handle in a manner that provides rotation thereof; and
an actuator carried by the handle, wherein the actuator is configured to affect rotation of the lens.

2. The lens handle assembly of claim 1, wherein the lens is surrounded by a collar, the collar defining a ring gear.

3. The lens handle assembly of claim 1, wherein the actuator includes a drive shaft having a drive gear disposed on the distal end thereof, the drive gear configured and arranged to mesh with the ring gear.

4. The lens handle assembly of claim 3, wherein the actuator is manually actuated.

5. The lens handle assembly of claim 3, wherein the actuator is actuated via a drive motor.

6. The lens handle assembly of claim 5, wherein the drive motor is mounted to the handle and interfaces with the drive shaft.

7. The lens handle assembly of claim 1, wherein the lens handle includes a handle portion and a lens retainer portion.

8. A lens handle assembly, comprising:
a handle;
a lens carried by the handle in a manner that provides rotation thereof; and
means for affecting rotation of the lens.

9. A method of viewing the ACA of a patient, the method comprising:
providing a lens handle assembly including a handle, a lens carried by the handle in a manner that provides rotation thereof, and an actuator carried by the handle, wherein the actuator is configured to affect rotation of the lens;
holding the handle of the lens handle with one hand; and
rotating the lens assembly with the one hand via the actuator.
